(19) United States
(12) Patent Application Publication
(10) Pub. No.: US 2003/0191064 A1
(43) Pub. Date: Oct. 9, 2003

(54) Methods for preventing and treating loss of balance function due to oxidative stress

(76) Inventor: Richard D. Kopke, San Diego, CA (US)

Correspondence Address: Counsel for Intellectual Property, Naval Medical Research Center, 503 Robert Grant Avenue, Silver Spring, MD, 20910-7500, US

(21) Appl. No.: 10/401,682

(22) Filed: Mar. 31, 2003

Related U.S. Application Data
(63) Continuation-in-part of application No. 09/766,625, filed on Jan. 23, 2001.

(51) Int. Cl. A61K 38/18; A61K 38/06; A61K 31/728; A61K 31/522; A61K 31/426; A61K 31/198; A61K 31/385; A61K 31/05; A61K 31/13

(52) U.S. Cl. 514/12; 514/18; 514/161; 514/263.31; 514/562; 514/440; 514/46; 514/369; 514/645; 514/733; 514/559; 514/563; 514/54

(57) ABSTRACT

The present invention provides methods for preventing and treating loss of, or impairments to, the sense of balance. Specifically, the invention provides methods for preserving the sensory hair cells and neurons of the inner ear vestibular apparatus by preventing or reducing the damaging effects of oxidative stress by administering an effective amount of the following therapeutic agents: antioxidants; compounds utilized by inner ear cells for synthesis of glutathione; antioxidant enzyme inducers; trophic factors; mitochondrial biogenesis factors; and combinations thereof.

### RELATED APPLICATIONS

This application is a continuation-in-part application of U.S. application Ser. No. 09/766,625 filed Jan. 23, 2001 (the entirety of which is incorporated herein by reference for all purposes) which claims benefit of Non-Provisional application Ser. No. 09/126,707, now U.S. Pat. No. 6,177,434 filed Jul. 31, 1998 (the entirety of which is incorporated herein by reference for all purposes) which claims benefit of provisional application No. 60/069,761 filed Dec. 16, 1997 (the entirety of which is incorporated herein by reference for all purposes).

### BACKGROUND OF THE INVENTION

1. Field of Invention

The present invention relates generally to methods and composition for preventing and treating loss of, or impairments to, the sense of balance. More specifically, the invention provides methods and composition for preserving the sensory hair cells and neurons of the inner ear vestibular apparatus by preventing or reducing the damaging effects of oxidative stress by administering an effective amount of certain therapeutic agents to a subject at risk for or experiencing loss of balance or injury to the inner ear balance organ.

2. Description of the Related Art

Balance dysfunction is a common disorder affecting as many as 40 million people in the United States per year. Dizziness is one of the most frequent complaints causing a patient to seek medical care (See Palaniappan R., Balance Disorders in Adults: An Overview, Hosp. Med. Vol., (2002) 63(5):278-81). Balance problems are a frequent cause of falls among the elderly and such falls are a common cause of death in this population (See Bloem B., Steijns J., and Smits-Engelsman B., An Update on Falls, Curr. Opin. in Neurol., (2003) 16(1):15-26). At this time, medical treatment for balance disorders consists of supportive therapy, treatment of symptoms, and surgical or medical ablation of the injured ear where dizziness symptoms are relieved by cutting the balance nerve or completely destroying the balance tissue. Currently, there is no clinically proven medical approach for balance disorders that is aimed at preventing or reversing injury to the inner ear balance system (See Brandt T., Management of Vestibular Disorders, J. Neurol. (2000) 247(7):491-9).

The cochlea and vestibular apparatus of the inner ear are both contained in a common bony labyrinth and surrounded by a common perilymphatic fluid. However, the tissue elements of the balance and hearing organs of the inner ear are anatomically distinct and have different neurosensory arrangements. Like the cochlea, the inner ear balance organs are composed of sensory hair cells, neurons, and supporting cells. Sensory hair cells transduce motion stimuli into a neuronal signal to the brainstem by releasing a chemical neurotransmitter such as glutamate into synaptic clefts between the hair cells and nerve endings (See Usami S., Takumi Y., Matsubara A., Fujita S., and Ottersen O., Neurotransmission in the Vestibular Endorgans-Glutamatergic Transmission in the Afferent Synapses of Hair Cells, Biol. Sci. Space, (2001) 15(4):367-70). These neural signals are transmitted to the brainstem and then to a variety of other central nervous system centers to provide sensory input essential for spatial orientation, balance, posture and locomotion.

The vestibular apparatus has three semicircular canals containing cristae which sense angular motion and two otolithic organs (the utricle and saccule) that sense linear acceleration including gravity. The cristae and otolithic organs are the repositories of the hair cells, supporting cells, dark cells, and neural synapse tissues. The balance organs of the inner ear are bathed in perilymphatic fluid in continuity with the cochlear tissues and the round window membrane (See Fritzsch B., Beisel K., Jones K., Farinas I., Maklad A., Lee J., and Reichardt L., Development and Evolution of Inner Ear Sensory Epithelia and their Innervation, J. Neurobiol. (2002) 53(2): 143-56). Thus, the cellular and molecular function of the inner ear balance organs is very similar to that of the cochlea, except that the cochlea transduces acoustic signals and the inner ear balance organs transduce movement signals presented to the organism.

Besides sharing common physiology, the cochlea and inner ear balance organs share a common pathophysiology. Many of the same things that damage the cochlea also damage the balance organs. In addition, the damage mechanisms and pathways are quite similar. For example, viruses (See Arbusow V. et al., HSV-1: Not Only in Human Vestibular Ganglia But Also in the Vestibular Labyrinth, Audiol. Neurootol. (2001) 6(5):259-62; Xie B. et al., Oxidative Stress in Patients with Acute Coxsackie Virus Myocarditis, Biomed. Environ. Sci. (2002) 15(1):48-57), bacterial infections (See Blank A., et al., Acute Streptococcus Pneumonide Meningogenic Labyrinthitis, Arch. Otol. Head & Neck Surg. (1994) 120:1342-1346; and Comis S., et al. Cytotoxic Effects on Hair Cells of Guinea Pig Cochlea Produced by Pneumolysin, the Thiol-Activated Toxin of Streptococcus Pneumoniae, Acta Otolaryngol (1993) 113(2):152-159), loud noise and acoustic trauma (See McCabe B., et al. The Effects of Intense Sound in the Non-Auditory Labyrinth, Acta Otolaryngol (1958) 49: 147-157; and Yilkoski J., Impulse Noise-Induced Damage in the Vestibular End Organs of the Guinea Pig, Acta Otolaryngol (1987) 103: 414-421), certain genetic disorders (See Gasparini P., et al. Vestibular and Hearing Loss in Genetic and Metabolic Disorders, Curr. Opin. Neurol. (1999) 12(1):35-9), and toxins (See Huang M. et al. Drug-Induced Ototoxicity: Pathogenesis and Prevention, Med. Toxicol. Adv. Drug Exp. 1989. 4(6):452-67), such as carbon monoxide and jet fuel fumes, may damage and destroy both cochlear and vestibular neurosensory tissue.

In addition, chemotherapy agents such as cisplatin (See Watanabe K., et al. Induction of Apoptotic Pathway in the Vestibule of Cisplatin (CDDP)-Treated Guinea Pigs, Anticancer Res. (2001) 21(6A):3929-32) and carboplatin (See Ding D., et al., Selective Loss of Inner Hair Cells and Type-I Ganglion Neurons in Carboplatin-Treated Chinchillas: Mechanisms of Damage and Protection, Ann. N.Y. Acad. Sci. (1999) 884:152-70) and certain antibiotics such as gentamicin and other aminoglycoside antibiotics damage the hair cells and neurons of the both the cochlea and vestibular apparatus (See Lang H., et al. Apoptosis and Hair Cell Degeneration in the Vestibular Sensory Epithelia of the Guinea Pig Following a Gentamicin Insult, Hearing Res. (1997) 111:177-184).

Aminoglycoside toxicity in the inner ear is different from other etiologies of oxidative stress in the inner ear because the aminoglycoside molecules react with iron in the inner ear to cause damage. Hence, for this particular class of toxins, iron-chelator therapy may prove to be effective (See Song B., et al. Iron-Chelators Protect from Aminoglycoside-Induced Cochleo- and Vestibulo-Toxicity, Free Radic. Biol. Med. (1998) 25(2):189-95).

Once the inner ear receives these stressful insults, common pathophysiological mechanisms for both the cochlea and vestibular tissues are activated. These mechanisms include the production of toxic free radical and reactive oxygen species (ROS) molecules (Takumida M., et al. Simultaneous Detection of Both Nitric Oxide and Reactive Oxygen Species in Guinea Pig Vestibular Sensory Cells, J. Otorhinolaryngol Relat. Spec. (2002) 64(2): 143-7.), excessive oxidative stress (generated by ischemia-reperfusion, glutamate excitotoxicity, and mitochondrial dysfunction and damage), and glutathione (GSH) depletion (See Ding D., et al., noted supra). These events lead to cellular injury and the activation of programmed cell death (PCD) (Watanabe K., et al., noted supra; Matsui J., et al., Inhibition of Caspases Prevents Ototoxic and Ongoing Hair Cell Death, J. Neurosci. (2002) 22(4): 1218-27; Ylikoski J., et al. Blockade of c-Jun N-terminal Kinase Pathway Attenuates Gentamicin-Induced Cochlear and Vestibular Hair Cell Death, Hear. Res. (2002) 163(1-2):71-81).

A real distinct advantage of the therapeutic compounds of the present invention (including antioxidants, antioxidants used by living cells to synthesize GSH, antioxidant enzyme inducers, trophic factors and mitochondrial biogenesis compounds) is that, for the most part, they are approved by the Food and Drug Administration, have extensive toxicology profiles, and are known to be very safe when given to humans. Specific antiapoptotic agents have been proposed in the literature to globally inhibit apoptosis throughout the entire organism (See Matsui J., et al., noted supra; and Ylikoski J. et al. also noted supra). These agents are not likely to be more effective than those described in the present invention and have the added risk of potentially being cancer causing since apoptosis is an important mechanism for the organism in preventing the development and spread of cancer (See Hayashi R., et al., Inhibition of Apoptosis in Colon Tumors Induced in the Rat by 2-amino-3-methylimidazo[4,5-f]quinoline, Cancer Res. (1996) 56(19):4307-10; Reed J., et al., BCL-2 Family Proteins: Regulators of Cell Death Involved in the Pathogenesis of Cancer and Resistance to Therapy, J. Cell Biochem. (1996) 60(1):23-32). In that regard, a distinct advantage of the present invention is the method of delivery of the agents of choice directly to the inner ear through the round window membrane, thus avoiding systemic side effects of the treatment agent. Additionally, when certain antiapoptotic agents are used, they may arrest the cell death processes temporarily but the cells remain in an injured and nonfunctional state (See Cheng A., et al. Calpain Inhibitors Protect Auditory Sensory Cells from Hypoxia and Neurotrophin-Withdrawal Induced Apoptosis, Brain Res. (1999) 850(1-2):234-43.)

Salicylate and other iron-chelators have been shown to be effective in preventing the ototoxicity associated with aminoglycoside antibiotics. The mechanism of damage with aminoglycoside antibiotics is unique in that it involves an interaction between the antibiotic and iron molecules in the inner ear. Salicylate and iron chelators are thus uniquely effective for aminoglycoside antibiotics. However, other stressors affecting the vestibular inner ear are not likely to involve an iron-mediated mechanism. Hence, salicylate by itself is not effective for noise damage to the inner ear (See Weisskopf P., Salicylate Decreases Noise-Induced Permanent Threshold-Shift, The American Academy of Otolaryngology Head and Neck Surgery, New Orleans, September 1999). Also, antioxidants that replenish glutathione are completely protective against cisplatin cochlear toxicity (See Campbell K., et al. D-methionine Provides Excellent Protection from Cisplatin Ototoxicity in the Rat, Hear Res. (1996) 102:90-8) whereas salicylate is only partially effective (Li G., Salicylate Protects Hearing and Kidney Function from Cisplatin Toxicity Without Compromising its Oncolytic Action, Lab Invest. 2002. 82(5):585-96). Thus, the present invention is superior to salicylate and iron chelators because it is effective for a wider variety of stressors not involving iron-mediated mechanisms and is more effective for cisplatin and noise stressors, for example.

Another proposed strategy to reduce the damage to the vestibular portion of the inner ear is through the inhibition of nitric oxide synthesis using nitric oxide synthesis inhibitors (See Takumida M., et al., noted supra). This approach was only partially effective in reducing oxidative stress. In addition, nitric oxide is an important second messenger for a wide range of cellular and molecular processes so that inhibiting the synthesis of NO is likely to cause unacceptable side effects (Fessenden JD and Schacht J. The Nitric Oxide/Cyclic GMP pathway: a potential major regulator of cochlear physiology, Hear Res. 1998. 118:168-76).

Thus, due to the common cellular and molecular biology, physiology, and pathophysiology, it has been found that the same treatments applicant set forth for the prevention and treatment of sensorineural hearing loss (presented in U.S. Pat. No. 6,177,434 and application Ser. No. 09/766,625 of which the present application is a continuation-in-part) will also be effective for the treatment of inner ear vestibular disorders.

### SUMMARY OF THE INVENTION

Accordingly, an object of this invention is to preserve the sensory hair cells of the inner ear vestibular apparatus by preventing the damaging effects of oxidative stress through the administration of an effective amount of therapeutic agents.

A further object of this invention is to preserve the sensory neurons of the inner ear vestibular apparatus by reducing the damaging effects of oxidative stress through the administration of an effective amount of therapeutic agents.

Yet another object of the invention is to provide methods for preserving the sensory hair cells and neurons of the inner ear vestibular apparatus through the administration of an effective amount of the following types of compounds: antioxidants, antioxidants used by living cells to synthesize GSH, antioxidant enzyme inducers, trophic factors, mitochondrial biogenesis compounds, and combinations thereof.

These and additional objects of the invention are accomplished by preventing or reducing the damaging effects of oxidative stress to sensory hair cells and neurons of the inner ear vestibular apparatus by administering:

antioxidants such as salicylic acid (including salt or ester), resveratrol, uric acid, as well as many other antioxidant compounds such as free radical spin trap agents such as phenyl-N-tert-butylnitrone;

antioxidants used by living cells to synthesize GSH or enhance GSH synthesis such as L-N-acetylcysteine, glutathione monoethyl ester (and other esters of glutathione), 1-2-oxothiazolidine-4-carboxylic acid (procysteine), L- and D-methionine, alpha-lipoic acid (and esters of alpha lipoic acid);

antioxidant enzyme inducers such as R-N6-phenylisopropyl adenosine;

trophic/growth factors such as brain-derived neurotrophic factor; nuerotrophin-3; epithelial growth factor (EGF) and the family of EGF growth factors including transforming growth factor alpha; insulin-like growth factor, and retinoic acid; and

mitochondrial biogenesis compounds such as acetyl-L-carnitine.

These agents can be used in combination and may be applied before, during or after balance disorder trauma. Also, this treatment has the potential to reverse balance disorders after they have occurred.

### BRIEF DESCRIPTION OF THE DRAWINGS

A more complete appreciation of the invention will be readily obtained by reference to the ‘Detailed Description of the Preferred Embodiments’ and these drawings.

FIGS. 1(A-C) depicts the data that demonstrate the protective effect of acetyl-Lcarnitine in preventing inner ear damage due to excessive noise exposure.

FIGS. 2(A and B) depicts data supporting the protective effect on balance function of trophic factors infused over the round window membrane of the inner ear after toxin induced inner ear damage. This is the first report we are aware of where this combination of trophic factors was able to restore balance function in an injury model using a novel technique of drug delivery to the round window membrane without violating the inner ear space.

FIGS. 3(A-C) depicts the data that demonstrate the protective effect of D-methionine in preventing inner ear damage due to excessive noise exposure.

FIG. 4 displays data supporting the efficacy of alpha-lipoic acid in preventing damage and permanent hearing loss due to excessive noise.

FIG. 5 portrays data supporting the efficacy of a combination of NAC and salicylate protecting the inner ear from noise damage. An optimal combination dosage was determined.

### DETAILED DESCRIPTION OF THE PREFERRED EMBODIMENTS

As used herein, the term “prevention”, in the context of the loss of or impairments to the sense of balance, death or injury of vestibular hair cells, death or injury of vestibular neurons, death or injury of vestibular dark cells and the like refers to minimizing, reducing, or completely eliminating the loss or impairment of balance function or damage, death or loss of those cells through the administration of an effective amount of one of the compounds described above in the present invention, ideally before an oxidatively stressful insult, or less ideally, shortly thereafter.

As used herein, the term “treatment”, refers to the administration of an effective amount of one of the compounds listed above to a subject who is experiencing loss or impairment of balance, or injury to or loss of vestibular hair cells, neurons, supporting cells, or dark cells, in order to minimize, reduce, or completely prevent or restore, the loss of balance function or hair cells, neurons or dark cells of the vestibular portion of the inner ear. Treatment is intended to also include the possibility of inducing, causing or facilitating regeneration of cellular elements of the inner ear including hair cells, supporting cells, dark cells, neurons and subcellular organelles of these cells including, synapses, stereocilia bundles, mitochondria and other cell organelles. Treatment is also intended to mean the partial or complete restoration of balance function irregardless of the cellular mechanisms listed above.

As used herein, the term “other stressors”, in the context of the loss of or impairments to the sense of balance is intended to include, but is not limited to: loud or excessive noise, trauma, toxins, ototoxic medications and drugs (other than aminoglycoside antibiotics), bacterial or viral infections, metabolic disorders or diseases, genetic disorders or diseases, and aging.

As used herein, the term “effective amount” refers to the amount of the compounds of the present invention required to achieve an intended purpose for both prophylaxis and treatment without unacceptable toxicity or undesirable side effects. It is synonymous herein with the term “pharmaceutically effective amount”. Optimal dose will depend on a number of factors such as route of administration (i.e. oral verses round window membrane delivery), constitutional factors of the patient, nature of the injury or potential injury, time from injury, and the nature and scope of the desired effect. Particular doses can be extrapolated from animal data and determined and adjusted by one skilled in the art. See infra for dose ranges of the present invention.

As used herein, the term “subject” refers to humans.

As used herein, “loss of or impairments to the sense balance”, “loss of balance function” and “balance disorders” are all terms that refer to a deficit in the vestibular system or vestibular function of a subject compared to the system of a normally functioning human. This deficit may completely or partially impair a subject's ability to maintain posture, spatial orientation, locomotion and any other functions associated with normal vestibular function.

As used herein, the term “administration” includes, but is not limited to, the following delivery methods: topical (including topical delivery to the round window membrane of the cochlea), oral, parenteral, subcutaneous, transdermal, and transbuccal administration.

As used herein, the term “damaging oxidative stress” refers to a state of stress on the organism, organs, tissues, or cells, especially but not limited to the inner ear, caused by an imbalance between oxidative chemical and biochemical molecules and reduced biochemical and chemical molecules, which results in impaired function or injury, either permanent or temporary to the organism. This state is characterized by the production of any of a variety of compounds known as free radicals or reactive oxygen species (ROS) and/or a reduction of any variety of any molecules in an organism known as antioxidants or that function as antioxidants. Damaging oxidative stress to the inner ear balance organ may arise through a number of different situations and hazard exposures including, but not limited to: loud noise, carbon monoxide, jet fuel fumes, industrial toxins, infectious agents such as bacteria and viruses, metabolic diseases such as diabetes mellitus, genetic disorders such as Connexin 26 disorder, exposure to chemotherapy agents (such as cisplatin, carboplatin and others), and the aging process.

As used herein “the inner ear balance organ” refers to the cells and tissues of the inner ear vestibular organ and inner ear vestibular labyrinth. These cells and tissues include but are not limited to, sensory hair cells (type I and type II), vestibular neurons, supporting cells, and dark cells of the vestibular system, the perilymphatic and endolymphatic fluids, tissues comprising the cristae of the three semicircular canals, and tissues comprising the otolithic organs (saccule and utricle).

In the present invention, applicants present methods for preventing and treating loss of balance function that includes selecting subjects experiencing said loss of balance function or at risk for acute exposure to noise, toxins, non-aminoglycoside antibiotics, medicines or other stressors causing said loss of balance function. After these individuals are selected, subjects are administered a pharmaceutically effective amount of one or more of the following agents: antioxidants; compounds utilized by inner ear cells for synthesis of glutathione; antioxidant enzyme inducers; trophic factors; and mitochondrial biogenesis factors. Antioxidants to be administered include salicylic acid, salts of salicylic acid, esters of salicylic acid, resveratrol, uric acid, and phenyl-N-tertbutylnitrone. Compounds utilized by inner ear cells for synthesis of glutathione to be administered include L-N-acetylcysteine, glutathione monoethyl ester, glutathione esters, 1-2-oxothiazolidine-4-carboxylic acid, L-methionine, D-methionine, alpha-lipoic acid, esters of alpha-lipoic acid. An example of an antioxidant enzyme inducer to be administered is R-N6-phenylisopropyl adenosine. Examples of trophic factors to be administered are brain-derived neurotrophic factor, nuerotrophin-3, epithelial growth factors, transforming growth factor alpha, insulin-like growth factor, retinoic acid. Finally, one example of a mitochondrial biogenesis factor to be administered is acetyl-L-carnitine.

Delivery of these therapeutic compounds to be administered in the present invention is accomplished by one or more of the following means: topical administration, topical administration to the round window membrane of the cochlea, oral administration, parenteral administration, subcutaneous administration, transdermal administration, transbuccal administration, and combinations thereof. Delivery of the compounds may also be accomplished by use of a catheter directed at or near the round window membrane and administering a solution that is compatible with and not toxic to the inner ear. A bio-compatible sustained delivery vehicle, such as fibrin glue or hyaluronic acid, can also be used to deliver these therapeutic compounds.

Because the cellular and molecular biology, physiology, and pathophysiology of the vestibular apparatus and the cochlea share many features in common, the treatment strategies developed by applicant for the cochlea (presented in U.S. Pat. No. 6,177,434 and application Ser. No. 09/766,625 (both of which are incorporated by reference herein)) have been shown to be effective for the balance system of the human inner ear. Such medical treatment will provide a significant advancement in treatment of balance disorders. This treatment is protective and/or restorative (not ablative) and is directed at the root cause of balance disorders, namely, the damage and death of vestibular hair cells and neurons, rather than providing just symptomatic treatment.

Thus, the same compounds that were described in U.S. Pat. No. 6,177,434 and application Ser. No. 09/766,625 (of which the present application is a continuation-in-part) and administered via oral preparation, intravenously, intramuscular injection, or topically to or near the round window membrane (RWM) as a solution through a catheter plant, will be effective in preventing and treating vestibular balance disorders of the inner ear in a way that preserves, restores and enhances balance function. The classes of agents used for the prevention and treatment of balance disorders in the present invention include:

antioxidants (such as salicylate)

antioxidants used by living cells to synthesize GSH or enhance GSH synthesis (such as N-acetylcysteine, methionine, glutathione monoethyl ester, alpha lipoic acid)

antioxidant enzyme inducers (such as R-N6-phenylisopropyl adenosine (R-PIA))

trophic factors (also known as growth factors)

mitochondrial biogenesis compounds (such as acetyl-L-carnitine)

The present invention shows that these therapeutic agents may also be effective if given systemically (at or near the RWM) or through other carrier vehicles such as fibrin glue or hyaluronic acid. A bio-compatible sustained delivery vehicle utilizing a solution, fibrin glue or hyaluronic acid is also an effective delivery method. The optimal method of delivery will depend on the particular compound being used and whether the compound is being used as a treatment or for prevention.

Preventive agents may best be administered orally prior to a predicted inner ear stress that could lead to damage. At other times, the preventive agent might best be administered more directly to the RWM in order to target the delivery of the medication to the inner ear. This latter approach can be advantageous in avoiding systemic side effects and complications associated with oral or intravenous or intramuscular administration of medications.

As an example, a number of these therapeutic compounds could be given orally to prevent the ototoxic effects of the cancer therapy agent cisplatin. However, these compounds administered to prevent the vestibular and cochlear damage caused by cisplatin, may also interfere with the cancer killing ability of the cisplatin. By delivering the inner ear protective agents only to the inner ear via the RWM, these compounds may be given in such a manner so as to avoid interfering with the cancer killing effects of the cisplatin while still preventing the ototoxicity.

The active compounds delivered topically to the RWM are able to pass through the RWM into the perilymphatic fluid for distribution to the injured vestibular neurosensory tissue. A detailed description of the preferred embodiments is found below. The common theme with all of these agents, compounds, or molecules is that they reduce the oxidative stress and oxidative stress injury so central in the pathophysiology of inner ear injury secondary to a wide variety of etiologies. Aging, toxins, noise, infections, genetic conditions, and side effects of a variety of medicines (such as aminoglycoside antibiotics and chemotherapy agents) can damage the inner ear leading to balance disorders. The present invention excludes prevention and treatment of loss of balance function due to aminoglycoside antibiotics because prior art is directed at this treatment (See Song B., et al. Iron-Chelators Protect from Aminoglycoside-Induced Cochleo- and Vestibulo-Toxicity, Free Radic. Biol. Med. (1998) 25(2):189-95).

A distinct advantage of the therapeutic compounds of the present invention (including antioxidants, antioxidants used by living cells to synthesize GSH, antioxidant enzyme inducers, trophic factors and mitochondrial biogenesis compounds) is: many are approved by the Food and Drug Administration, have extensive toxicology profiles, and are known to be very safe when given to humans. Another clear advantage of the present invention is the method of delivery of the agents of choice directly to the inner ear through the round window membrane, thus avoiding systemic side effects of the treatment agent. In addition, the present invention is superior to salicylate and iron chelators because it is effective for a wider variety of stressors not involving iron-mediated mechanisms.

Another proposed strategy to reduce the damage to the vestibular portion of the inner ear is through the inhibition of nitric oxide synthesis using nitric oxide synthesis inhibitors (See Takumida M., et al., noted supra). This approach was only partially effective in reducing oxidative stress. In addition, nitric oxide is an important second messenger for a wide range of cellular and molecular processes so that inhibiting the synthesis of NO is likely to cause unacceptable side effects.

Classes of Therapeutic Compounds for the Present Invention:

a. Antioxidants

These compounds include salicylic acid (including salt or ester), resveratrol, uric acid, as well as many other antioxidant compounds such as free radical spin trap agents such as phenyl-N-tert-butylnitrone (PBN) and other antioxidants. These antioxidant agents are effective by scavenging the toxic free radical and ROS generated by the insult induced on the inner ear and thereby reduce the amount of damage sustained by the inner ear. This reduces the damage and loss of hair cells and neurons in the vestibular neuroepithelium.

b. Antioxidants Used by Living Cells to Synthesize GSH

This subset of antioxidant compounds include L-N-acetylcysteine, glutathione monoethyl ester (and other esters of glutathione), 1-2-oxothiazolidine-4-carboxylic acid (Procysteine), L- and D-methionine, alpha-lipoic acid (and esters of alpha lipoic acid), and many others in this class. These compounds are free radical scavengers but have the additional beneficial property of being broken down and resynthesized into intracellular glutathione (GSH). Alpha-lipoic acid enhances the re-synthesis of reduced GSH. GSH depletion is likely a key pathologic event in most of the injury processes of the inner ear. GSH replenishment can prevent and reverse some of the acute damage associated with acoustic over exposure and toxins such as cisplatin and amino-glycoside antibiotics (See Kopke R., et al. Enhancing Intrinsic Cochlear Stress Defenses to Reduce Noise-Induced Hearing Loss. Laryngoscope (2002) 112:1515-32).

c. Antioxidant Enzyme Inducers

This category of compounds includes adenosine agonists such as R-N6-phenylisopropyl adenosine (R-PIA). R-PIA has been shown to increase or induce the activity of antioxidant enzymes in the inner ear rendering the inner ear more resistant to noise damage and damage from certain toxins (See Kopke R., Use of Organotypic Cultures of Corti's Organ to Study the Protective Effects of Antioxidant Molecules on Cisplatin-induced damage of auditory hair cells, Am. J. Otol. (1997) 18(5):559-71; and Hu B., et al R-phenylisopropyladenosine Attenuates Noise-Induced Hearing Loss in the Chinchilla, Hearing Research. 113(1-2):198-206).

d. Trophic Factors

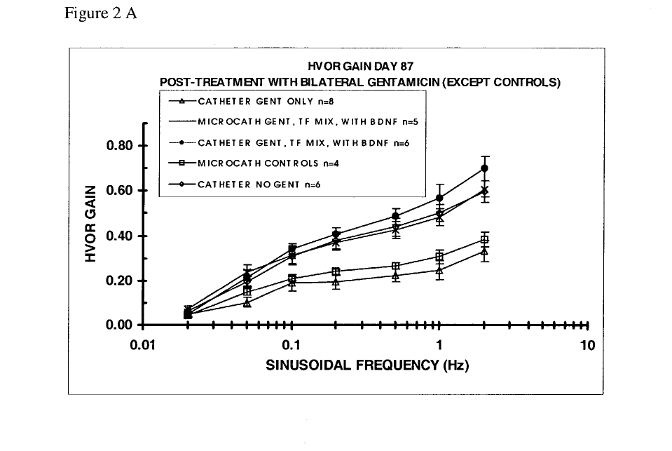

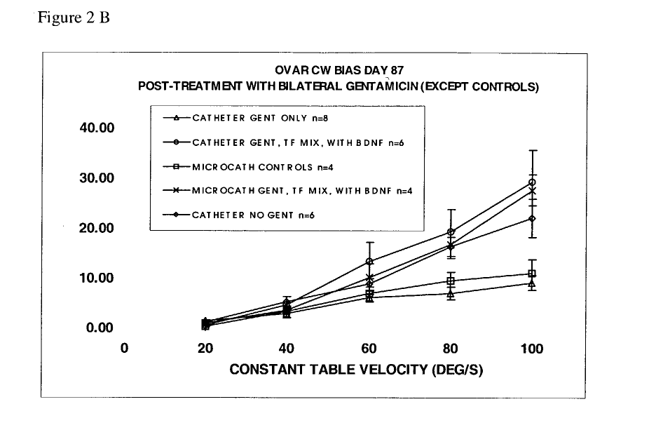

Polypeptide trophic factors (also known as growth factors) can reduce inner ear injury by halting the programmed cell death (PCD) pathway processes thereby inhibiting cell death and sparing the loss of hair cells and neurons to preserve function. Trophic factors have also been shown to prevent injury, cell death, and loss of function by increasing the activity of antioxidant defenses (See Jonas C., et al. Keratinocyte Growth Factor Enhances Glutathione Redox State in Rat Intestinal Mucosa During Nutritional Repletion, J. Nutr. (1999) 129(7):1278-84). Examples of these trophic factors used in the present invention include: Brain Derived Neurotrophic Factor (BDNF); Neurotrophin-3 (NT-3); Epithelial Growth Factor (EGF) and the family of EGF growth factors including transforming growth factor alpha (TGF-alpha); Insulin-Like Growth Factor (IGF-1); retinoic acid; and many others common growth factors. While the use of trophic factors to ameliorate vestibular injury has been disclosed (See Kopke R., et al. Growth Factor Treatment Enhances Vestibular Hair Cell Renewal and Results in a Recovery of Function, PNAS (2001) 98(10):5886-5891.), one embodiment of the present invention utilizes noninvasive delivery of these compounds to the round window membrane of the inner ear. This noninvasive method for delivery of medications to the inner ear has a distinct advantage for preserving or restoring inner ear balance function. Invasive strategies involve drilling through the bone of the inner ear labyrinth and are apt to be fraught with unacceptable damage to the inner ear. This embodiment aspect of the present invention is elaborated in FIG. 2 and Example two, infra.

e. Mitochondrial Biogenesis Compounds

Acetyl-L-carnitine is the prototypical compound in this category. Acetyl-L-carnitine restores mitochondrial integrity and function for mitochondria injury secondary to oxidative stress (See Kopke R., et al. Enhancing Intrinsic Cochlear Stress Defenses to Reduce Noise-Induced Hearing Loss, Laryngoscope (2002) 112:1515-32). By restoring mitochondrial function and reversing mitochondrial injury due to oxidative stress, injury to the cells of the inner ear vestibular apparatus can be reduced.

f. Use of Combinations of Compounds to Prevent or Treat the Loss of Balance

The common underlying pathophysiologic process involved with injury to the inner ear resulting in loss of balance is oxidative stress. The organism has a number of strategies for countering oxidative stress including intrinsic small molecule antioxidants, the synthesis and utilization of glutathione, antioxidant enzymes, trophic factors and mitochondrial biogenesis factors. These redundant systems for ameliorating oxidative stress are more effective together than by themselves as each strategy has a distinct mechanism and cellular and biochemical pathway associated with it (See Eisen A., et al. Treatment of Amyotrophic Lateral Sclerosis, Drugs Aging. (1999) 14(3): 173-96). Accordingly, the present invention discloses the use of combinations of the above listed compounds (Classes a through e) when such combinations are more effective than the individual compounds in preventing or treating the loss of the sense of balance. An example of the use of a combination of agents follows (see figure five and example five).

The dose ranges for the present invention are listed below.

Antioxidants

Salicylate

Systemic—1-100 mg/kg/day

Antioxidants that are Used by Living Cells to Synthesize GSH

N-acetylcysteine (NAC)

Systemic—0.1-150 mg/kg/day

Round window membrane delivery—0.0001-15 mg/kg/day

Methionine

Systemic—0.1-150 mg/kg/day

Round window membrane delivery—0.0001-15 mg/kg/day

Glutathione Monoethyl Ester

Systemic—0.1-150 mg/kg/day

Round window membrane delivery—0.0001-15 mg/kg/day

Alpha Lipoic Acid

Systemic—0.1-150 mg/kg/day

Round window membrane delivery—0.0001-15 mg/kg/day

Antioxidant Enzyme Inducers

R-N6-phenylisopropyl Adenosine (R-PIA)

Round window membrane delivery—0.0001-5 mg/kg/day

Trophic Factors (Includes all Trophic Factors Listed Above for Dose Ranging)

Round window membrane delivery—0.00001-10 mg/kg/day

Mitochondrial Biogenesis Compounds

Acetyl-L-carnitine

Systemic—0.1-150 mg/kg/day

Round window membrane delivery—0.0001-15 mg/kg/day

### EXAMPLES

Having described the present invention, the following examples are given to illustrate specific applications of the invention including the best mode now known to perform the invention. These specific examples are not intended to limit the scope of the invention described in this application.

### Example 1

### Prevention of Inner Ear Function and Hair Cell Loss with Acetyl-L-carnitine

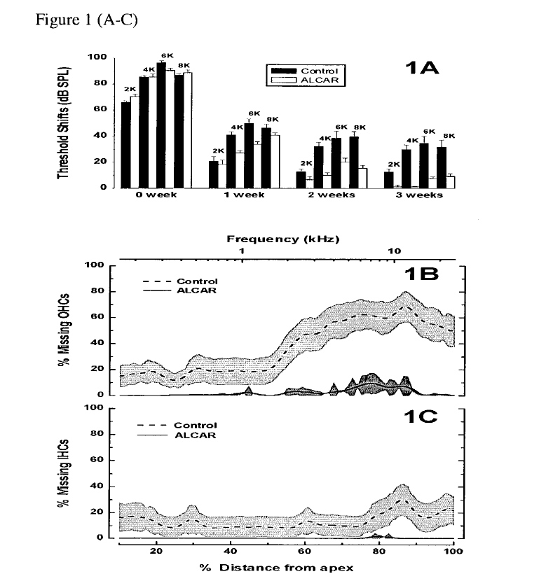

In this example, the inner ear stress was loud noise. Chinchilla (n=6 per group) were exposed to six hours of continuous 4 kHz octave band noise at 105 dB SPL. Treated animals received intraperitoneal injections of a solution of acetyl-L-carnitine (ALCAR), 100 mg/kg, in sterile normal saline and control animals received sterile normal saline injections alone. The injections were given every 12 hours starting 48 hours before the noise exposure, 1 hour before noise, 1 hour after noise, then twice per day for the next two days. Hearing thresholds at 2, 4, 6, and 8 kHz were measured in the animals before noise exposure, 1 hour after noise exposure, and each week for three weeks using a commonly accepted method (auditory brainstem response or ABR). After the final hearing tests the animals were humanely euthanized and the cochleae were harvested so that inner ear hair cells that remained viable could be counted using well-accepted methodology. Results are depicted in FIGS. 1(A-C). ALCAR treatment significantly reduced permanent hearing loss as seen by the reduced threshold shifts noted at the three-week time point (p<0.01) as seen in FIG. 1A. ALCAR treatment also resulted in a highly significant reduction in inner (IHCs) and outer (OHCs) loss in the cochlea (p<0.01) as seen in FIGS. 1B and 1C.

Thus, this example illustrates successful protection of inner ear hair cells from a common oxidative stressor, namely loud noise. Overall, a dramatic reduction in lost function and hair cell loss was achieved. As discussed supra, this experimental data and results can be directly extrapolated to use in balance disorder therapies with similarly anticipated beneficial results.

### Example 2

### Recovery of Balance Function Loss Due to the Aminoglycoside Antibiotic Gentamicin by Topical Treatment Using Trophic Factors in Guinea Pig

In this example, it was shown that inner ear balance function was permanently reduced by exposing guinea pigs to gentamicin, an ototoxic antibiotic. The gentamicin was placed in the middle ear space so as to diffuse into the inner ear across the round window membrane. One week later, guinea pigs were treated topically in one ear with a solution of growth factors consisting of TGF-α (2 μgm/ml), IGF-1 (200 ngm/μl), RA (10⁻⁸M), and BDNF (1 mg/ml). One group of animals received an infusion of carrier vehicle only into the inner ear. Another group received the carrier vehicle with the trophic factors through a catheter placed directly into the inner ear through a hole drilled into the bony labyrinth. The final group had the same trophic factor treatment but the trophic factors were delivered onto the round window membrane of the inner ear (microcatheter).

This is a unique and important aspect of the present invention as it represents the first report of the successful restoration of balance function by applying a trophic factor solution to the round window membrane. This less invasive procedure than catheter infusion into the inner ear is an important aspect of the instant invention in that it makes such treatment of the inner ear practical and possible in humans. Delivery of medication into the human inner ear by drilling a hole into the labyrinth is apt to cause an unacceptable level of damage to the inner ear.

Inner ear balance function was later measured several months after the gentamicin exposure and trophic factor treatment. As shown in FIG. 2A, there was a complete recovery of a measure of horizontal semicircular canal function known as the horizontal vestibulo-ocular reflex (HVOR) in the trophic factor treated animals. The HVOR function in the animals treated by the round window membrane application of trophic factors was as good as the function of the animals that had direct infusion through a catheter placed through a hole drilled into the inner ear and was significantly better than the untreated control HVOR function (p<0.01) as seen in FIG. 2A. Another aspect of balance function governed by the utricle and saccule, OVAR bias, was also significantly improved (p<0.01) by both trophic factor treatments as seen in FIG. 2B. As discussed supra, this experimental data and results can be directly extrapolated to use in balance disorder therapies with similarly anticipated beneficial results.

### Example 3

### Prevention of Inner Ear Function and Hair Cell Loss with D-methionine (MET)

In this example, the inner ear stress was loud noise. Chinchilla (n=6 per group) were exposed to six hours of continuous 4 kHz octave band noise at 105 dB SPL. Treated animals received intraperitoneal injections of a solution of MET (200 mg/kg) in sterile normal saline and control animals received sterile normal saline injections alone. The injections were given every 12 hours starting 48 hours before the noise exposure, 1 hour before noise, 1 hour after noise, then twice per day for the next two days. Hearing thresholds at 2, 4, 6, and 8 kHz were measured in the animals before noise exposure, 1 hour after noise exposure, and each week for three weeks using a commonly accepted method (auditory brainstem response or ABR). After the final hearing tests the animals were humanely euthanized and the cochleae were harvested so that inner ear hair cells that remained viable could be counted using well-accepted methodology.

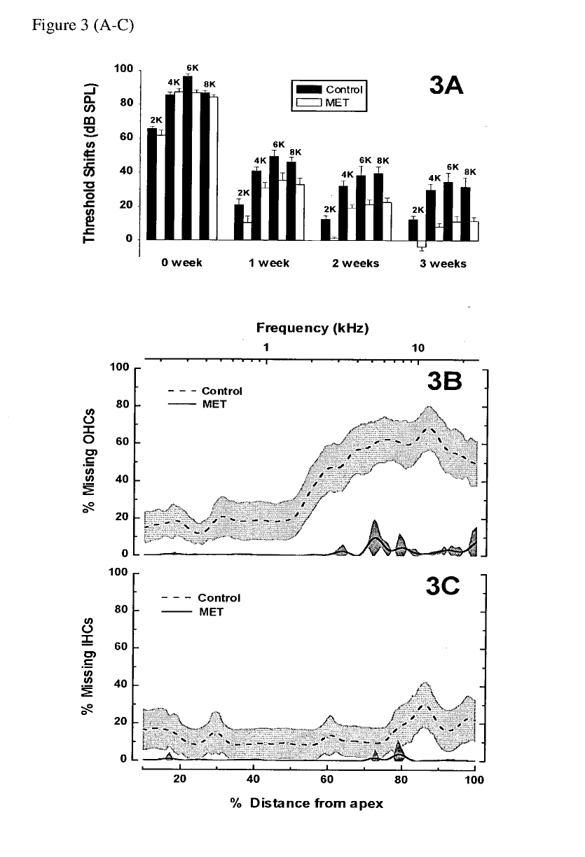

Results are depicted in FIGS. 3(A-C). MET treatment significantly reduced permanent hearing loss as seen by the reduced threshold shifts noted at the three-week time point (p<0.01) as seen in 3A. MET treatment also resulted in a highly significant reduction in inner (IHCs) and outer (OHCs) loss in the cochlea (p<0.01) as seen in 3B and 3C. Thus, this example illustrates successful protection of inner ear hair cells from a common oxidative stressor, namely loud noise. Overall, a dramatic reduction in lost function and hair cell loss was achieved. As discussed supra, this experimental data and results can be directly extrapolated to use in balance disorder therapies with similarly anticipated beneficial results.

### Example 4

### Prevention of Inner Ear Function & Hair Cell Loss with Alpha Lipoic Acid (LA)

In this example, the inner ear stress was loud noise. Chinchilla (n=6 per group) were exposed to six hours of continuous 4 kHz octave band noise at 105 dB SPL. Treated animals received intraperitoneal injections of a solution of LA (100 mg/kg) in sterile normal saline and control animals received sterile normal saline injections alone. The injections were given every 12 hours starting 7 days before the noise exposure, 1 hour before noise, 1 hour after noise, then twice per day for the next two days. Hearing thresholds at 2, 4, 6, and 8 kHz were measured in the animals before noise exposure, 1 hour after noise exposure, and each week for three weeks using a commonly accepted method (auditory brainstem response or ABR).

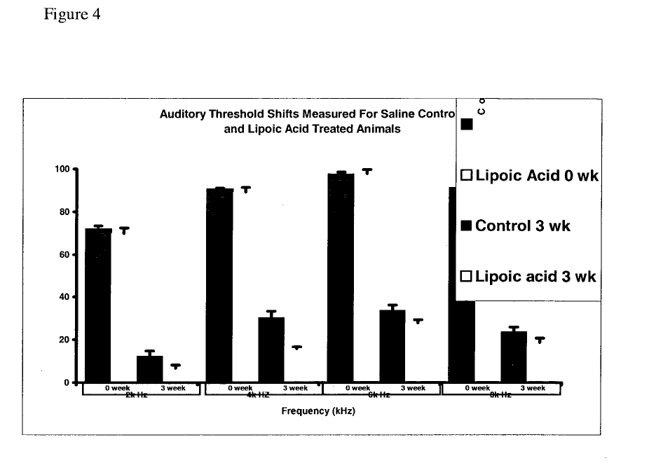

LA treatment significantly reduced permanent hearing loss as seen by the reduced threshold shifts noted at the three-week time point (p<0.01) as seen in FIG. 4. Thus this example illustrates successful protection of inner ear function from a common oxidative stressor, namely loud noise. Overall, a significant reduction in lost function was achieved. As discussed supra, this experimental data and results can be directly extrapolated to use in balance disorder therapies with similarly anticipated beneficial results.

### Example 5

### Prevention of Inner Ear Damage with a Combination of Antioxidants

In this example, the inner ear stress was loud noise. Chinchilla (n=6 per group) were exposed to six hours of continuous 4 kHz octave band noise at 105 dB SPL. Treated animals received intraperitoneal injections of a solution of N-acetylcysteine (NAC), 325 mg/kg, and one of three doses of salicylate (25, 50, or 75 mg/kg) in sterile normal saline and control animals received sterile normal saline injections alone. The injections were given every 12 hours starting 48 hours before the noise exposure, 1 hour before noise, 1 hour after noise, then twice per day for the next two days. Hearing thresholds at 2, 4, 6, and 8 kHz were measured in the animals before noise exposure, 1 hour after noise exposure, and each week for three weeks using a commonly accepted method (auditory brainstem response or ABR).

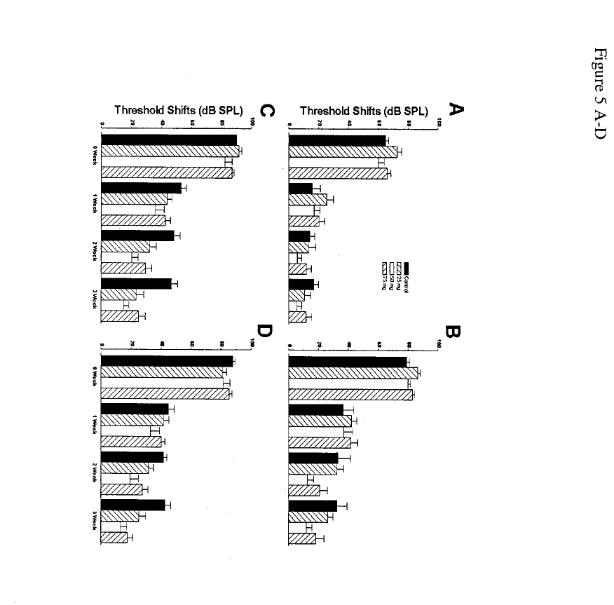

NAC and salicylate treatment significantly reduced permanent hearing loss as seen by the reduced threshold shifts noted at the three-week time point (p<0.001) as seen in FIG. 5. Thus, this example illustrates successful protection of inner ear function from a common oxidative stressor, namely loud noise. Overall, a significant reduction in lost function was achieved. This example further illustrates how combinations of antioxidants may be more effective in reducing inner ear damage as seen with the significantly better hearing threshold recovery for the group receiving NAC plus 50 mg/kg of salicylate (p<0.01 for 4K and 8K ABR threshold shifts 3 weeks after noise exposure comparing 50 mg/kg and 25 mg/kg salicylate plus NAC). In addition NAC plus 25 mg/kg salicylate was only better than control at 6 kHz whereas the NAC plus 50 mg/kg was better than control at three weeks at all four frequencies. As discussed supra, this experimental data and results can be directly extrapolated to use in balance disorder therapies with similarly anticipated beneficial results.

### DETAILED DESCRIPTION OF THE FIGURES

FIG. 1A. Chinchilla were exposed to 6 hours of 4 kHz octave band noise at 105 dB SPL. Noise-induced hearing threshold shifts (hearing loss) from chinchilla are depicted for four frequencies over three weeks. Animals given intraperitoneal (IP) injections of acetyl-L-carnitine (ALCAR) shortly before and after the loud noise exposure had significantly less hearing loss at all frequencies compared to untreated noise-exposed controls. (n=6 animals, error bars are standard error of mean, statistical analyses shows a significant treatment effect 3 weeks after noise exposure (2-way ANOVA, using treatment and frequency as dependent variables, p<0.01)

FIGS. 1B&C. Depicted are cytocochleogram data. These are records of outer (1B) and inner (1C) hair cell loss as a function of anatomic location within the cochleae of animals whose hearing thresholds are depicted in 1A. The hair cell counts were obtained after the final hearing tests were performed three weeks after the noise exposure. There was substantially less outer hair cell (OHC) and inner hair cell (IHC) loss observed for the ALCAR treated animals compared to the untreated noise exposed controls. (n=12 ears for each group, the shaded area represents the standard error of means. 2-way ANOVA comparing the OHC's of controls and ALCAR treated chinchillas showed a significant improvement when animals were treated with ALCAR (p<0.01). A similar comparison for IHC's shows significant reduction for saline-treated animals (controls, p<0.05)

FIG. 2A. Guinea pigs were given an administration of gentamicin that reduced (triangles and squares) a measure of balance function known as horizontal vestibuloocular gain (HVOR gain). This balance function measure records how well eye motion is coordinated with head motion. HVOR gain is a measure of semicircular canal function in the inner ear. The semicircular canals sense any angular velocity of the head. One week after gentamicin exposure treated animals received a solution of growth factors through a catheter inserted directly into the inner ear (catheter) or through a microcatheter placed on the round window membrane (microcath). The trophic factor (TF) solution consisted of BDNF, TGF alpha, IGF-1, and retinoic acid. Both groups of animals receiving the trophic factor solution had a substantial and statistically significant recovery of HVOR gain (circles and Xs) that was similar to control values from animals not exposed to gentamicin (diamonds). The recovery was to the level of control animals not damaged by gentamicin (data not shown). What is quite unique about these data is that the recovery induced by the trophic factors delivered to the round window membrane was as good as the recovery induced by a direct and invasive injection into the inner ear. Note: error bars are standard error of means, statistical analysis was performed by 2-way ANOVA (dependent factors are treatment and HVOR frequency), with Fisher's Least Squares Differences Post-hoc test. HVOR gain recovery in the TF treated groups was significantly better than gentamicin only controls (p<0.01).

FIG. 2B. Guinea pigs were given an administration of gentamicin that reduced (triangles and squares) a measure of balance function known as off vertical axis rotation (OVAR) bias. This balance function measure records how well eye motion is coordinated with head motion. OVAR bias is a measure of otolithic function in the inner ear. One week after gentamicin exposure treated animals received a solution of growth factors through a catheter inserted directly into the inner ear (catheter) or through a microcatheter placed on the round window membrane (microcath, a less invasive procedure than the catheter insertion). The trophic factor (TF) solution consisted of BDNF, TGF alpha, IGF-1, and retinoic acid. Both groups of animals receiving the trophic factor solution had a substantial and statistically significant recovery of OVAR bias (circles and Xs) similar to control values from animals not exposed to gentamicin (diamonds). The recovery was to the level of control animals not damaged by gentamicin (data not shown). What is quite unique about these data is that the recovery induced by the trophic factors delivered to the round window membrane was as good as the recovery induced by a direct and invasive injection into the inner ear. Note: error bars are standard error of means, statistical analysis was accomplished by 2-way ANOVA (dependent factors are treatment and OVAR table speed), with Fisher's Least Squares Differences Post-hoc test. OVAR bias recovery in the TF treated groups was significantly better than gentamicin only controls (p<0.01).

FIG. 3A. Chinchilla were exposed to 6 hours of 4 kHz octave band noise at 105 dB SPL. Noise-induced hearing threshold shifts (hearing loss) from chinchilla are depicted for four frequencies over three weeks. Animals given intraperitoneal (IP) injections of D-methionine (MET) shortly before and after the loud noise exposure had significantly less hearing loss at all frequencies compared to untreated noise-exposed controls. (n=6 animals, error bars are standard error of mean, statistical analysis showed a significant treatment effect for all frequencies tested (P<0.01).

FIGS. 3B&C. Depicted are cytocochleogram data. These are records of outer (1B) and inner (1C) hair cell loss as a function of anatomic location within the cochleae of animals whose hearing thresholds are depicted in 1A. The hair cell counts were obtained after the final hearing tests were performed three weeks after the noise exposure. There was substantially less outer hair cell (OHC) and inner hair cell (IHC) loss observed for the MET treated animals compared to the untreated noise exposed controls. (n=12 ears for each group, the shaded area represents the standard error of means, statistical analysis show a significant reduction in both OHC's (p<0.01) and IHC's (p<0.05)).

FIG. 4. Chinchilla were exposed to 6 hours of 4 kHz octave band noise at 105 dB SPL. Noise-induced hearing threshold shifts (hearing loss) from chinchilla are depicted for four frequencies one hour and three weeks after the noise exposure. Animals given intraperitoneal (IP) injections of alpha lipoic acid (lipoic acid) shortly before and after the loud noise exposure had significantly less hearing loss at all frequencies compared to untreated noise-exposed controls who received saline injections rather than lipoic acid. (n=6 animals, error bars are standard error of mean, statistical analysis using 2-way repeated measures analysis of variance showed a statistically significant treatment effect p<0.01 and Fisher's LSD post hoc testing revealed a significant reduction in hearing loss at each frequency, p<0.05).

FIG. 5. Threshold Shifts. These panels demonstrate the dose response curves utilizing different concentrations of salicylate with a constant dose of NAC (325 mg/kg). Panels A, B, C, and D represent sensitivities at frequencies of 2, 4, 6, and 8 kHz respectively. All groups showed some improvement up to 2 weeks, and then only the LNAC/salicylate groups continued to improve. The 50 mg/kg salicylate group demonstrated a statistically significant improvement at all frequencies compared to controls at the 3 weeks (ANOVA p<0.001, Newmann-Kuels post hoc test). These data demonstrate that an optimal combination of NAC plus salicylate affords optimal inner ear protection from loud noise. The best protection is seen with the NAC salicylate combination where the salicylate is 50 mg/kg (p<0.01 for 4K and 8K ABR threshold shifts 3 weeks after noise exposure comparing 50 mg/kg and 25 mg/kg salicylate plus NAC).

What is claimed:

1. A method for preventing and treating loss of balance function comprising:
selecting subjects experiencing said loss of balance function or at risk for acute exposure to noise, toxins, non-aminoglycoside antibiotics, medicines or other stressors causing said loss of balance function; and
delivery to said subjects a pharmaceutically effective amount of an agent selected from the group consisting of:
antioxidants;
compounds utilized by inner ear cells for synthesis of glutathione;
antioxidant enzyme inducers;
trophic factors;
mitochondrial biogenesis factors; and
combinations thereof.

2. The method of claim 1, wherein said antioxidant is selected from the group consisting of salicylic acid, salts of salicylic acid, esters of salicylic acid, resveratrol, uric acid, phenyl-N-tert-butylnitrone, and combinations thereof.

3. The method of claim 1, wherein said compounds utilized by inner ear cells for synthesis of glutathione is selected from the group consisting of L-N-acetylcysteine, glutathione monoethyl ester, glutathione esters, 1-2-oxothiazolidine-4-carboxylic acid, L-methionine, D-methionine, alpha-lipoic acid, esters of alpha-lipoic acid, and combinations thereof.

4. The method of claim 1, wherein said antioxidant enzyme inducer is R-N6-phenylisopropyl adenosine.

5. The method of claim 1, wherein said trophic factor is selected from the group consisting of brain-derived neurotrophic factor, nuerotrophin-3, epithelial growth factors, transforming growth factor alpha, insulin-like growth factor, retinoic acid, and combinations thereof.

6. The method of claim 1, wherein said mitochondrial biogenesis factor is acetyl-L-carnitine.

7. The method of claim 1, wherein said delivery is accomplished by a means that is selected from the group consisting of topical administration, topical administration to the round window membrane of the cochlea, oral administration, parenteral administration, subcutaneous administration, transdermal administration, transbuccal administration, and combinations thereof.

8. The method of claim 1, wherein said delivery is accomplished via a catheter directed at or near the round window membrane and administering a solution of said agent that is compatible with and not toxic to the inner ear.

9. The method of claim 1, wherein said delivery is accomplished by incorporation of said agent in a bio-compatible sustained delivery vehicle.

10. The method of claim 9, wherein said biocompatible carrier vehicle is selected from the group consisting of fibrin glue and hyaluronic acid.
